(19) United States
(12) Patent Application Publication
(10) Pub. No.: US 2024/0215877 A1
(43) Pub. Date: Jul. 4, 2024

(54) A gel-based matrix for extending biocatalyst turnover lifetime

(71) Applicant: Milo Sensors, Inc., Santa Barbara, CA (US)

(72) Inventors: Peter Lamberg, Santa Barbara, CA (US); Rashad Hamid, Santa Barbara, CA (US); Bob M Lansdorp, Santa Barbara, CA (US)

(21) Appl. No.: 18/398,794

(22) Filed: Dec. 28, 2023

Related U.S. Application Data
(60) Provisional application No. 63/477,465, filed on Dec. 28, 2022.

(51) Int. Cl. A61B 5/1486; A61B 5/00

(52) U.S. Cl. CPC A61B 5/1486; A61B 5/4845; A61B 2562/125

(57) ABSTRACT

A device for maintaining functionality in a device reliant on active biocatalysts. A sensor has a biological catalyst, a buffered gel, and a detector component. The gelling component of the buffered gel can be adjusted to function as a barrier or semi-permeable membrane to keep the enzyme active longer. Experimental results show that the invention can sustain device activity for at least 5 days, which is normally only active for up to 1 day.

This application claims priority from Provisional application No. 63/477,465; filed Dec. 28, 2022, the entire contents of which are herewith incorporated by reference.

This invention was made with government support under R44AA026125, awarded by the National Institute of Health (NIH). The government has certain rights in the invention.

### BACKGROUND

### Field of the Invention

The present invention is related to retaining enzyme activity in the presence of deactivating molecules.

### Description of Related Art

Biological catalysts have been investigated extensively over the past decades due to their substrate specificity. Enzymes may be used to catalyze reactions with nearly every known biomarker and, when coupled to a sufficient detection system, power highly specific diagnostic devices. A major challenge for most enzyme-based sensing technologies is low stability, both shelf life and lifetime in active state. A common way to keep such units fresh is to refrigerate, freeze or lyophilize batches and dissolve them immediately before use.

The necessity of refrigerated storage challenges the commercial viability of a portable biosensor based on biocatalysts. Commonly used enzymatic biosensors have strict storage conditions that may extend shelf life substantially. However, once such sensors are taken out of their storage structures, most cannot be used more than once since enzymes usually deactivate much more rapidly once they react with their substrate. Moreover, many enzymes are relatively expensive to procure and stabilize. As a result, enzymatic sensors typically take on a disposable form factor using expensive cartridges that can only perform one round of analysis.

In a state-of-the-art wearable alcohol biosensor (Lansdorp et al, Sensors, 2019), enzyme was fully hydrated within a short period of time, and could continuously measure the presence of alcohol. However, the sensor was observed to stop functioning after 24 hours, due to the loss of enzyme activity.

The inventors recognized the need for an integrated solution to bypass rapid enzyme deactivation in a bioelectronic device. Having an integrated system is especially useful in wearable enzyme-based monitoring devices.

Prior art mentions the use of immobilization protocols and molecular modulation to improve the longevity of a sensing device using enzymes, most often including the use of aldehydes and other crosslinkers (US20170009270A1). However, many biocatalysts are either inhibited by such modifications or completely deactivated. Therefore, the inventors recognize the need for a means of extending the operational life of enzyme-based devices without chemically modifying the enzyme.

Prior art that uses barriers intended specifically for amperometric glucose sensors mention bodily fluids as the main deactivating factor (CN209878659U). However, they fail to acknowledge that their Ag/AgCl reference electrode is also a major contributor to enzyme deactivation.

Furthermore, many studies have investigated the immobilization of enzymes, such as alcohol oxidase and glucose oxidase, as a means of improving stability. For example, when authors entrapped glucose oxidase in a film of poly(acrylonitrile-co-acrylic acid), improved stability was claimed (D. Shan et al., A porous poly(acrylonitrile-co-acrylic acid) film-based glucose biosensor constructed by electrochemical entrapment, Analytical Biochemistry 356:2, 2006). However, the improved stability referred to shelf life and environmental factors. No evidence was presented as to the stability during continuous operation of the amperometric sensor. Furthermore, no evidence was presented of a Silver or Silver-chloride poisoning effect that we teach here.

### SUMMARY

This invention describes a structure that protects a sensitive biocatalyst from toxic or deactivating compounds in a biosensing device.

### BRIEF DESCRIPTION OF THE DRAWINGS

FIGS. 1A, 1B and 1C show schematic representations of different views of the enzymatic electrochemical sensor used in amperometric readings of transdermal alcohol;

FIG. 2 shows a graph of residual enzyme catalytic activity when stored in a neutral buffer with a buffer strength of 10 mM vs 100 mM;

FIG. 3 shows a graph of pesidual activity of enzyme stored in a test tube, enzyme stored in the whole sensing compartment, and enzyme stored in a test tube with the isolated Ag/AgCl reference electrode;

FIGS. 4A-4D shows graphs of Amperometric setups containing the same amount of active enzyme under constant turnover conditions; where FIG. 4A compares with buffered 0.25% Agarose gel, FIG. 4B with buffered 15% Sodium Polyacrylate, FIG. 4C buffered 15% Crosslinked Sodium Polyacrylate, and FIG. 4D compares with dialysis membrane covering the electrode with phosphate buffer;

FIG. 5 shows a graph of amperometric sensing under constant flow of ethanol with 3% vs 15% gel NaPA gel;

FIG. 6 shows a flow of a drying and rehydration process of a hygroscopic gel with an enzymatic biocomponent.

### DETAILED DESCRIPTION

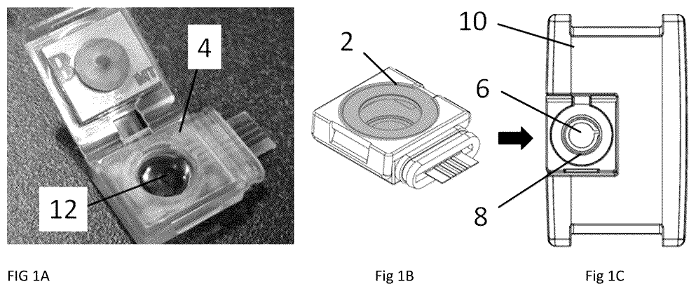

In one embodiment shown in FIGS. 1A, 1B and 1C, the biosensor is an amperometric alcohol sensor based on a dry deposit of stabilized alcohol oxidase.

The biosensor contains a semi-permeable membrane (2), sensor compartment (4), prussian blue electrode (6), and silver/silver chloride (Ag/AgCl) reference electrode (8). Prior to activation, the biosensor is kept dry and inactive until use for optimal shelf life. Once the device is connected to an external electronic amperometric reader (10) circuit, the semipermeable membrane 2 is placed in contact with the skin of the user, for example by wearing the system as a wearable wristband. Ethanol from the blood of the user's skin diffuses passively through the skin as insensible perspiration or actively as sensible perspiration, through the membrane 2, and reaches the sensor surface.

Once the biosensor compartment (cartridge, 4) is hydrated, for example with a phosphate buffered saline solution 12 or other liquid familiar to those skilled in the art, Alcohol Oxidase is dissolved and becomes active, allowing an operating window of up to 24 hours before the enzyme degrades beyond a usable state. This operating window where the enzyme is active can be extended by adding a larger enzyme deposit. For example, Lansdorp et al (Lansdorp, Bob, et al. “Wearable enzymatic alcohol biosensor.” Sensors 19.10 (2019): 2380) showed that alcohol oxidase stays active for at least 12 hours in continuous catalyzing. The inventors recognize the desirability of a means of extending the operating window to multiple days of continuous operation, with minimal impact on sensor performance, ideally with no increase in enzyme amount.

Experiments were performed to measure alcohol oxidase activity using an ABTS assay that relies on peroxidase and a color-changing dye familiar to those skilled in the art (Verduyn, Corenelis, Johannes P. van Dijken, and W. Alexander Scheffers. “Colorimetric alcohol assays with alcohol oxidase.” Journal of Microbiological Methods 2.1 (1984): 15-25.) to determine the activity of alcohol oxidase.

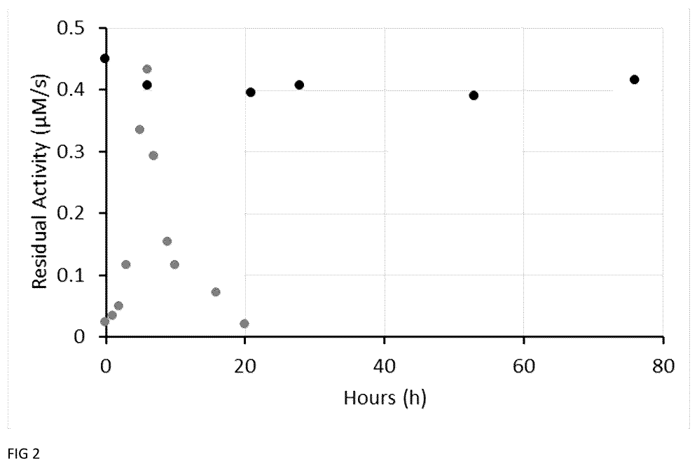

In FIG. 2, residual enzyme activity readings towards ethanol using 2,2′-azino-bis(3-ethylbenzothiazoline-6-sulfonic acid) (ABTS) were compared for enzyme stored in 10 mM and 100 mM buffer strength. The enzyme in dilute buffer retained over 50% activity for 8 hours. Surprisingly, the 100 mM buffer kept a nearly constant activity over at least 3 days, indicating that the concentration of buffering molecules around the alcohol oxidase octamers is critical to the stability of the enzyme.

The above experiment was performed on enzyme kept in small plastic tubes only containing phosphate buffer and potassium chloride.

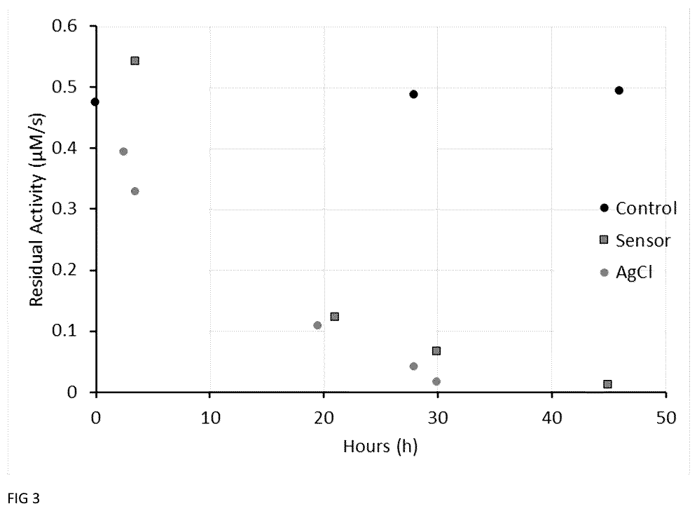

However, once the enzyme-buffer combination was stored in the biosensor cartridge, the enzyme stability was once again dropping to around 12 hour halftime when using the ABTS activity assay (shown in FIG. 3, as the gray squares). This effect was seen while simply using the isolated cartridge as a storage container with no reader connected and no alcohol present, using an ABTS assay. This result indicated that despite adequate buffer strength, one or more components in the sensor cartridge deactivated the enzyme prematurely. Through performing careful control experiments, a surprising discovery was made: that the enzyme deactivated much quicker by being stored in the same plastic tube as a Ag/AgCl electrode (FIG. 3, gray circles). Thus, while the enzyme in the control experiment lasted for 48 hours, by incubating enzyme in the presence of a Ag/AgCl electrode the stability was reduced to a half-life of approximately 12 hours.

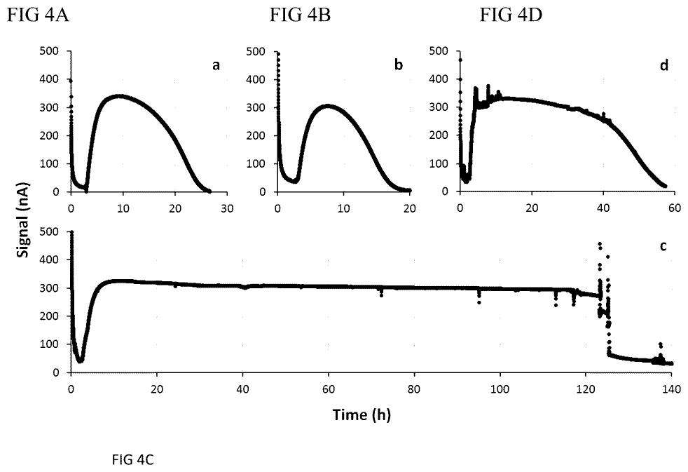

In FIGS. 4A, 4B, 4C and 4D, different approaches were attempted in order to keep Alcohol Oxidase from making contact with deactivating components. Gels are known to maintain pore sizes relative to the polymer type, crosslinking, and concentration, as is familiar to those skilled in the art (Primera, J., Hasmy, A. & Woignier, T. Numerical Study of Pore Sizes Distribution in Gels. Journal of Sol-Gel Science and Technology 26, 671-675 (2003)). Activity experiments were performed using an amperometric electrochemical measurement (10) containing Alcohol Oxidase. An applied potential of +93 mV on the working electrode (6) with respect to the pseudo-reference AgCl (8) was used in amperometric measurement system familiar to those skilled in the art. Gels of 35 microLiters were made directly on the working electrode surface (6), and stabilized alcohol oxidase was hydrated and pipetted on. A flow-cell was used to flow ethanol solution of 25 mM at a constant flow rate over a diffusion-limiting membrane as has been previously described (Lansdorp, Bob, et al. “Wearable enzymatic alcohol biosensor.” Sensors 19.10 (2019): 2380). In the case of a 0.25% Agarose gel (FIG. 4a), the sensor retains activity towards ethanol for approximately 12 hours. Similarly, in the case of buffered 15% Sodium PolyAcrylate (FIG. 4b), the enzyme retains activity for 12 hours. However, in the case where 15% Crosslinked Sodium Polyacrylate (cNaPA) is used, the sensor retains activity for more than 120 hours (FIG. 4c). Remarkably, in this case the enzyme is in the presence of AgCl which has been shown herein to be a destabilizing agent. Thus, the addition of a 15% cNaPA has prolonged the sensor activity, while 15% non-crosslinked dispersed PA does not.

The precise mechanism of the extended enzyme activity may be due to scavenging of the silver, scavenging of the silver chloride, stabilizing of the alcohol oxidase enzyme, or through a filtration effect, or through another effect. Regardless of the precise mechanism of sensor stabilization, it has nonetheless been discovered that a cross-linked PolyAcrylate Gel (12) can stabilize an enzyme-based biosensor, even in the presence of destabilizing agents such as Ag/AgCl.

It is known in the literature that polyacrylate is a silver scavenger, meaning that the improvement may have been due to a scavenging effect (Boris G. Ershov and Arnim Henglein. Reduction of Ag⁺ on Polyacrylate Chains in Aqueous Solution The Journal of Physical Chemistry B. 1998 102 (52), 10663-10666). Polyacrylate can be used to form silver nanoparticles in a silver solution due to this phenomenon. Furthermore, enzyme activity has been shown to be improved through the addition of chelating agents (Berberich, Jason A., et al. “A stable three enzyme creatinine biosensor. 2. Analysis of the impact of silver ions on creatine amidinohydrolase.” Acta Biomaterialia 1.2 (2005): 183-191.). However, the results indicated that addition of 15% sodium polyacrylate alone did not have any noticeable improvement on the operating window (FIG. 4c). This indicates that the crosslinking is critical to the continued operation of the device. By deduction, this suggests that silver/silver chloride scavenging was an unlikely mechanism by which the sensor lifetime improved.

A longer operating window could also be seen while using a dialysis membrane (Bartovation, VDT01R10 12-14 kDa) to cover the bottom of the sensor cartridge (4) containing the two electrodes (6,8) (FIG. 4d). The buffer and dissolved Alcohol Oxidase deposited atop the membrane. The dialysis membrane had a pore size of 12-14 kDa, which could catch particles as small as 2 nm. However, a dialysis membrane has a limited lifetime, and will eventually swell and deform. This may explain why it could not reach the stabilities consistently exceeding three days, as seen in the experiments with cNaPA. Moreover, the dialysis membrane is very fragile and difficult to hermetically seal. The reason why the operating window was not improved further could be related to the swelling/deforming nature of the membrane. It was proved that the element responsible for deactivating the enzyme was most likely AgCl crystals dissociating from the reference electrode (8), seeing that silver ions would have freely diffused through the membrane.

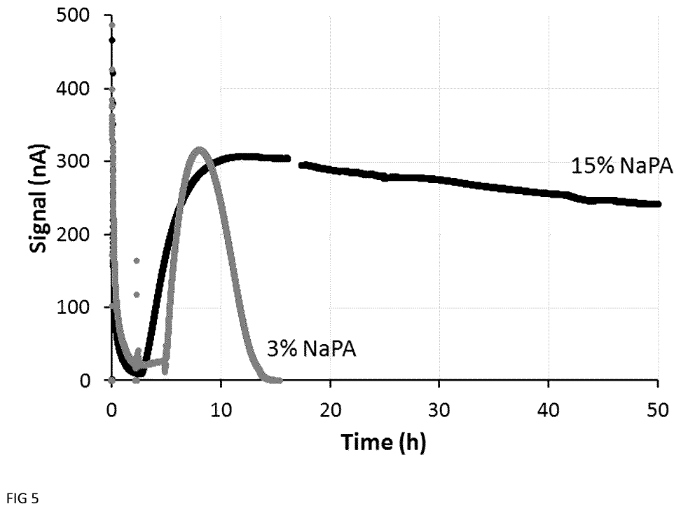

Through a series of experiments, it was found that the crosslinked NaPA gel needed to be of a high weight percentage. When a lower concentration of gel was used in an amperometric sensor no improvement in the operational window was observed (FIG. 5). A gel concentration of 5% or more was required to improve stability.

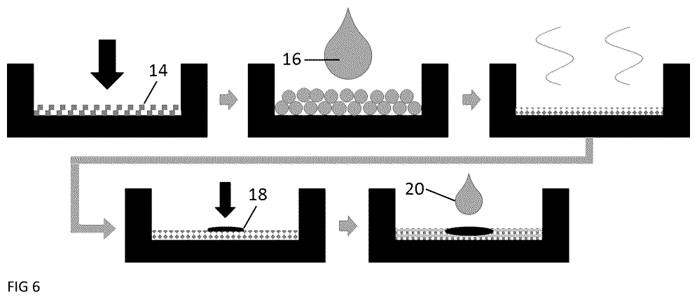

While 15-50% cNaPA with 100 mM phosphate buffer can successfully prevent deactivation in an amperometric biosensor, the resulting gel is highly granular and difficult to accurately dispense once prepared. A method to accurately obtain a gel layer with 35 microLiter liquid buffer was developed (FIG. 6). 5 mg of dry gel, here, NaPA (14) was measured and added to the sensor compartment (4) that includes the biosensor. 50 microLiters of ultrafiltrated water (16) was added to the compartment (4), swelling the hygroscopic gel particles, thus temporarily hydrating the particles. The resulting gel was evaporated at elevated temperature (30° C.) until fully dry, forming a coarse layer of dry NaPA particles fused to the sensor floor, which forms a dry and rough film.

Dry stabilized enzyme (18) was dispersed in DI water, added atop the coarse layer and dried at 30° C. under a flow of argon or nitrogen. The resulting dry layer could be transported to an end user where the final activation step is performed; addition of 35 microLiters of 100 mM buffer (20).

Although only a few embodiments have been disclosed in detail above, other embodiments are possible and the inventors intend these to be encompassed within this specification.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An enzymatically catalyzed sensing device comprising:
A biological catalyst promoting a reaction of a substrate or analyte,
A buffered gel, having an aqueous buffer with a gelling component; and
An interfacing detector sensitive to a product of biological catalysis.

2. The device of claim 1, where the biological catalyst is an enzyme.

3. The device of claim 1, wherein the biological catalyst is a flavin-containing oxidase enzyme.

4. The device of claim 1, where the buffered gel also includes a supporting electrolyte.

5. The device of claim 1, where the buffered gel has a buffer concentration which is greater than 40 mM.

6. The device of claim 1, where the gelling component of the buffered gel is comprised of at least one polymer.

7. The device of claim 1, where the gelling component of the buffered gel includes polymers in a form of cross-linked hygroscopic particles.

8. The device in claim 6, where the polymer is cross-linked Sodium Polyacrylate.

9. The device in claim 8, where a polymer concentration is higher than 3%.

10. The device in claim 9, where the polymer concentration is higher than 10%.

11. The device of claim 1, wherein the interfacing detector is an amperometric sensor comprised of two or more electrodes.

12. The device in claim 11, where one electrode is a silver/silver chloride electrode.

13. The device in claim 12, where the biological catalyst is an enzyme of alcohol oxidase.

14. The device in claim 13, wherein the sensing device is a transdermal alcohol sensor.

15. The device of claim 1, wherein the interfacing detector contains or interfaces with a wearable device.

16. A method for creating a user-activated biosensing device comprising:
Adding dry gel to a compartment containing a biosensor;
Adding liquid to temporarily hydrate particles of the dry gel, to form a hydrated gel;
Dehydrating the hydrated gel until a dry and rough film is formed;
Rehydrating with a buffer to a final volume to make an active catalytic device.

17. The method of claim 16, where the dry gel includes cross-linked sodium polyacrylate.

18. The method in claim 16, where a stabilized form of biocatalyst is added to a sensor compartment before said rehydrating.

19. The method of claim 18, where the biocatalyst is alcohol oxidase.

20. The method of claim 16 where the liquid is primarily water.

21. The method of claim 16, where a lyophilized alcohol oxidase biocatalyst is hydrated and subsequently dried before activation.

22. The method of claim 16, wherein the biosensor is an amperometric detection device.

23. The method of claim 16, wherein the biosensor contains alcohol oxidase for transdermal alcohol detection.
